(19) United States
(12) Patent Application Publication
(10) Pub. No.: US 2024/0374384 A1
(43) Pub. Date: Nov. 14, 2024

(54) PROSTHETIC HEART VALVE HAVING FRAME WITH VARYING STRUT LENGTHS

(71) Applicant: EDWARDS LIFESCIENCES CORPORATION, Irvine, CA (US)

(72) Inventors: Anatoly Dvorsky, Haifa (IL); Nikolai Gurovich, Hadera (IL); Michael Bukin, Pardes Hanna (IL); Eran Grosu, Kfar-Saba (IL)

(21) Appl. No.: 18/780,203

(22) Filed: Jul. 22, 2024

Related U.S. Application Data
(63) Continuation of application No. PCT/US2023/011165, filed on Jan. 19, 2023.
(60) Provisional application No. 63/302,310, filed on Jan. 24, 2022.

(51) Int. Cl. A61F 2/24

(52) U.S. Cl. CPC A61F 2/2418; A61F 2/2436; A61F 2230/0069; A61F 2250/0037

(57) ABSTRACT

A prosthetic heart valve can include a radially expandable and compressible frame and a valvular structure. The frame can include a plurality of circumferentially spaced axial struts, a plurality of pairs of axial posts, and a plurality of rows of angled struts. Each pair of axial posts can be positioned circumferentially between two axial struts. The plurality of rows can include at least a first row, a second row downstream of the first row, a third row downstream of the second row, and a fourth row downstream of the third row. Each of the angled struts can be connected at one end to one of the axial struts and at another end to one of the axial posts. The angled struts of the first and fourth rows can be shorter than the struts of the second and third rows.

### CROSS-REFERENCE TO RELATED APPLICATIONS

The present application is a continuation of PCT Application No. PCT/US2023/011165, filed Jan. 19, 2023, which claims the benefit of U.S. Provisional Application No. 63/302,310, filed Jan. 24, 2022, which is incorporated herein by reference in its entirety.

### FIELD

The present disclosure relates to a design for an implantable, expandable prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

When mechanical actuators are incorporated in the frame of the prosthetic valve, the frame may be subject uneven or excessive forces, which can cause portions of the frame to twist, rotate, or otherwise deform when the prosthetic heart valve is deployed to the expanded configuration. Accordingly, there exists a need to increase the stability of the prosthetic heart valve during the valve's expansion.

### SUMMARY

A frame for a mechanically expandable and compressible prosthetic heart valve in accordance with certain examples of the invention can solve one or more deficiencies in the prior art. In particular, the frame is configured to prevent elements of the frame from twisting or rotating when the valve deployed to an expanded configuration, thereby increasing the stability of the prosthetic heart valve.

A prosthetic heart valve can comprise a frame and a valvular structure coupled to the frame. In addition to these components, a prosthetic heart valve can further comprise one or more of the components disclosed herein.

In some examples, the frame is radially expandable and radially compressible.

In some examples, the frame comprises a plurality of groups of four outer struts and two axial struts forming a plurality of hexagonal outer cells arranged side-by-side in a circumferential direction of the frame, and a plurality of groups of four inner struts forming a plurality of diamond-shaped inner cells, wherein each inner cell is located within a respective outer cell, wherein the outer struts are shorter than the inner struts.

In some examples, the inner struts have a first uniform length.

In some examples, the outer struts have a second uniform length less than the first uniform length.

In some examples, the outer struts are curved.

In some examples, the frame comprises an inflow end and an outflow end, each outer cell comprises an inflow apex disposed towards the inflow end and an outflow apex disposed towards the outflow end, and for each outer cell, a first pair of two of the outer struts are connected to the inflow apex, and a second pair of two of the outer struts are connected to the outflow apex.

In some examples, for each outer cell, each outer strut of the first pair comprises a curved portion adjacent the inflow apex and each outer strut of the second pair comprises a curved portion adjacent the outflow apex.

In some examples, each inner cell comprises an inflow apex disposed towards the inflow end and an outflow apex disposed towards the outflow end, and for each inner cell, a first pair of two of the inner struts are connected to the inflow apex of the inner cell, and a second pair of two of the inner struts are connected to the outflow apex of the inner cell.

In some examples, the frame comprises a plurality of pairs of first and second axially extending posts, wherein the first and second posts of each pair are axially spaced from each other, and wherein each pair of first and second posts is positioned circumferentially between two of the axial struts.

In some examples, for each inner cell, the inner struts of the first pair of inner struts are connected to a first axially extending post of a pair of first and second posts, and the inner struts of the second pair of inner struts are connected to a second axially extending post of the pair of first and second posts.

In some examples, for each outer cell, the outer struts of the first pair of outer struts are connected to a first axially extending post of a pair of first and second posts, and the outer struts of the second pair of outer struts are connected to a second axially extending post of the pair of first and second posts.

In some examples, the prosthetic heart valve comprises a plurality of actuator members, each extending through a pair of first and second axially extending posts.

In some examples, the valvular structure comprises a plurality of leaflets forming a plurality of commissures, wherein each commissure is secured to one of the axial struts.

In some examples, the frame is cylindrical.

In a representative example, a prosthetic heart valve can comprise a radially expandable and compressible frame. The frame can comprise a plurality of groups of four outer struts and two axial struts forming a plurality of hexagonal outer cells and a plurality of groups of four inner struts forming a plurality of diamond-shaped inner cells. Each inner cell can be located within a respective outer cell and the outer struts are shorter than the inner struts. The prosthetic heart valve can further comprise a valvular structure disposed within the frame, wherein the valvular structure can be configured to regulate the flow of blood through the frame in one direction.

In another representative example, a prosthetic heart valve can a include a radially expandable and compressible frame. The frame can include a plurality of circumferentially spaced axial struts and a plurality of pairs of axial posts, wherein each pair of axial posts can be positioned circumferentially between two axial struts. In such examples, the frame can further comprise a plurality of rows of angled struts, including at least a first row, a second row downstream of the first row, a third row downstream of the second row, and a fourth row downstream of the third row, wherein each angled strut can be connected at one end to an axial strut and at another end to an axial post. In such examples, the struts of the first and fourth rows can be shorter than the struts of the second and third rows. In such examples, the frame can further include a valvular structure that can be disposed within the frame and can be configured to regulate the flow of blood through the frame in one direction.

In another representative example, a prosthetic heart valve comprises a radially expandable and compressible frame comprising a plurality of cell columns arranged in a circumferentially extending row of cell columns, wherein each cell column comprises an outer cell and an inner cell disposed within the outer cell, wherein each inner cell comprises four angled inner struts and each outer cell comprises four angled outer struts, wherein the outer struts are shorter than the inner struts. The prosthetic valve can further comprise a valvular structure disposed within the frame and configured to regulate the flow of blood through the frame in one direction.

In some examples, a prosthetic heart valve comprises one or more of the components recited in Examples 1-34 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2A is a perspective view of a frame for the prosthetic valve of FIG. 1A.

FIG. 2B is a front portion of the frame shown in FIG. 2A.

FIG. 3 is a side elevation view of a delivery apparatus for a prosthetic device, such as a prosthetic valve, according to one example.

FIG. 4 is a perspective view of a portion of an actuator of the prosthetic device of FIGS. 1-2 and an actuator assembly of a delivery apparatus, according to one example.

FIG. 5 is a perspective view of the actuator and actuator assembly of FIG. 4 with the actuator assembly physically coupled to the actuator.

FIG. 6 is a perspective view of a portion of a frame for a prosthetic valve, according to another example.

FIG. 7A is a side elevation view of an inner strut of the frame shown in FIG. 6.

FIG. 7B is a side elevation view of an outer strut of the frame shown in FIG. 6.

FIG. 8 is a perspective view of a frame for a prosthetic valve shown in a flattened configuration, according to another example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

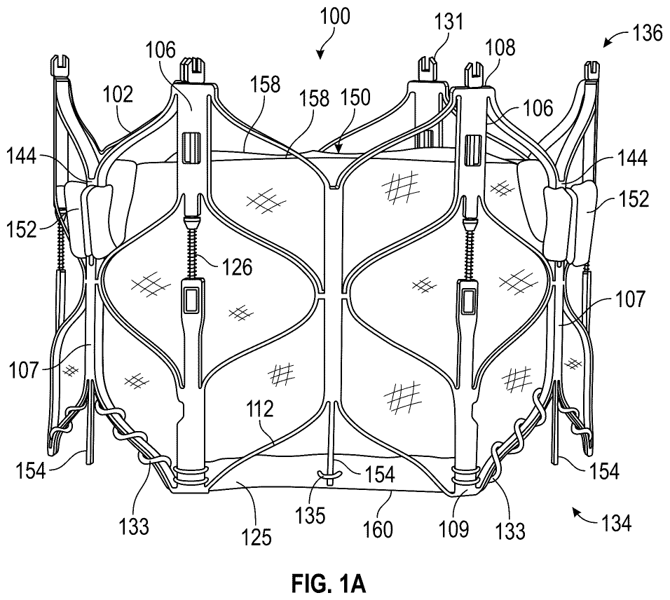

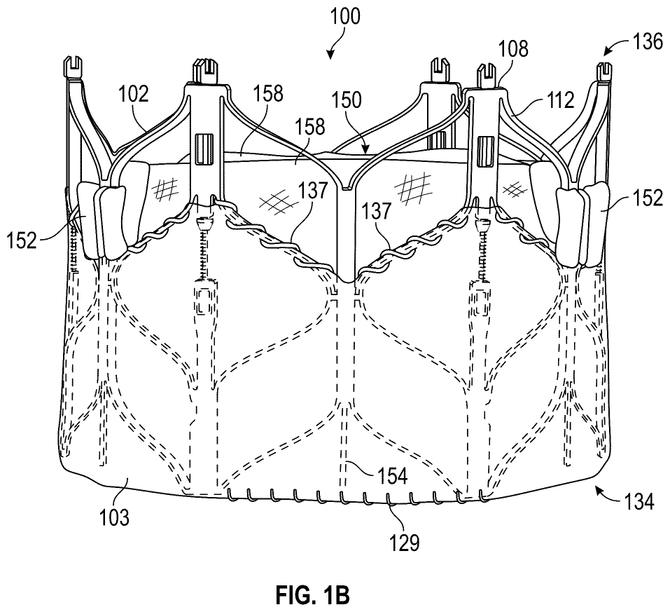

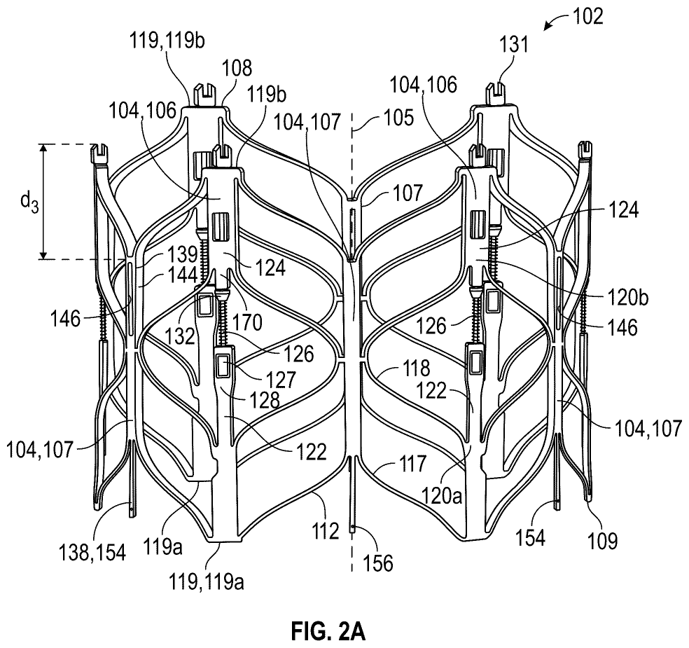

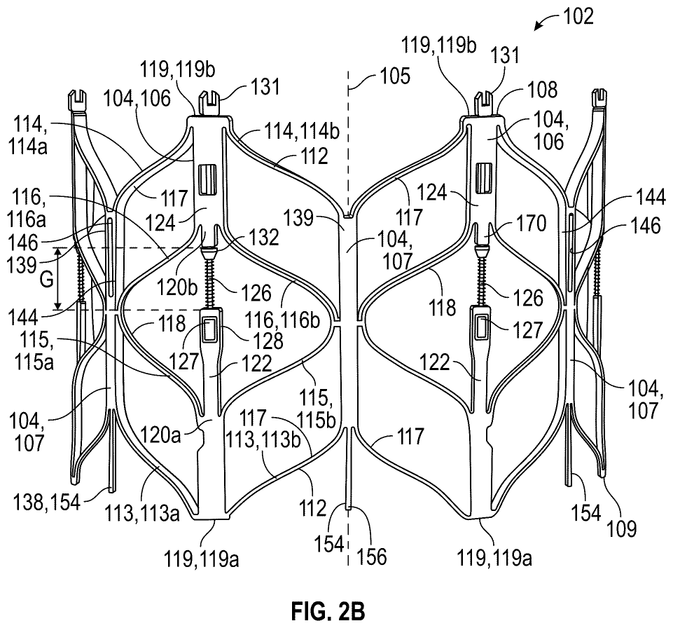

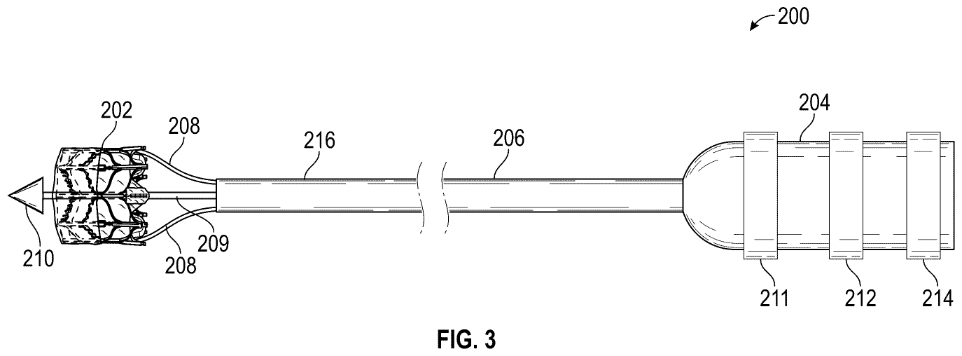

FIGS. 1A-2B illustrate an exemplary prosthetic device (for example, prosthetic heart valve) that can be advanced through a patient's vasculature, such as to a native heart valve, by a delivery apparatus, such as the exemplary delivery apparatus shown in FIG. 3. The frame of the prosthetic heart valve can include one or more mechanical expansion and locking mechanisms that can be integrated into the frame-specifically, into axially extending posts of the frame. The mechanical expansion and/or locking mechanisms can be removably coupled to, and/or actuated by, the delivery apparatus to radially expand the prosthetic heart valve and lock the prosthetic heart valve in one or more radially expanded states.

### Examples of the Disclosed Technology

FIGS. 1A-2B show an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-2B illustrate an exemplary example of a prosthetic valve 100 (which also may be referred to herein as “prosthetic heart valve 100”) having a frame 102. FIGS. 2A-2B show the frame 102 by itself, while FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103. While only one side of the frame 102 is depicted in FIG. 2B, it should be appreciated that the frame 102 forms an annular structure having an opposite side that is substantially identical to the portion shown in FIG. 1B, as shown in FIGS. 1A-2A.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a scaling member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In other examples, as shown in FIG. 1A, the leaflets 158 can be sutured directly to the frame 102 or to a reinforcing member 125 (also referred to as a reinforcing skirt or connecting skirt) in the form of a strip of material (for example, a fabric strip) which is then sutured to the frame 102, along the scallop line via stitches (for example, whip stitches) 133.

The inner and outer skirts and the connecting skirt 125 can be formed from any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valve can be found, for example, in U.S. Patent Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102, which is shown alone and in greater detail in FIGS. 2A and 2B, comprises and inflow end 109, an outflow end 108, and a plurality of axially extending posts 104. In particular examples, the frame 102 in its radially expanded configuration can have a cylindrical shape having a constant diameter from the inflow end 109 to the outflow end 108. The axial direction of the frame 102 is indicated by a longitudinal axis 105, which extends from the inflow end 109 to the outflow end 108 (FIGS. 2A and 2B). Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In other examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (i.e., each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in other examples.

As illustrated in FIG. 2B, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (i.e., openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIGS. 2A and 2B, each second cell 118 can be disposed within one of the first cells 117 (i.e., the struts 115a-116b forming the second cells 118 are disposed between the struts forming the first cells 117 (i.e., the struts 113a, 113b and the struts 114a, 114b), closer to an axial midline of the frame 102 than the struts 113a-114b).

As illustrated in FIGS. 2A and 2B, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and twelve posts 104. However, in other examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap G (FIG. 2B) (those with actuators 126 can be referred to as actuator posts or actuator struts). Each first post 122 (i.e., the lower post shown in FIGS. 2A and 2B) can extend axially from the inflow end 109 of the prosthetic valve 100 toward the second post 124, and the second post 124 (i.e., the upper post shown in FIGS. 2A and 2B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In other examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In other examples, not all pairs of posts 122, 124 need be expansion and locking mechanisms (i.e., actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap G (FIG. 2B) separating the posts 122, 124, and thereby radially compressing or radially expanding the prosthetic valve 100, respectively. Thus, the gap G (FIG. 2B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (for example, the delivery apparatus 200 of FIG. 3, as described below). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (for example, in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame. When the threaded rod 126 is rotated in the first direction, the head portion 131 of the rod 126 bears against an adjacent surface of the frame (for example, an outflow apex 119b), while the nut 127 and the first post 122 travel proximally along the threaded rod 126 toward the second post 124, thereby radially expanding the frame. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 108 of the frame until the stopper 132 abuts the inflow end 170 of the second post 124 (as shown in FIGS. 2A and 2B). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (for example, counterclockwise) causing the stopper 132 to push against (i.e., provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In other examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIGS. 2A and 2B, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (for example, a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some examples, the commissure opening 146 can be fully enclosed by the support post 107 such that a portion of the valvular structure 150 can be slid radially through the commissure opening 146, from an interior to an exterior of the frame 102, during assembly. In the illustrated example, the commissure opening 146 has a substantially rectangular shape that is shaped and sized to receive commissure tabs of two adjacent leaflets therethrough. However, in other examples, the commissure opening can have any of various shapes (for example, square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance d₃ (indicated in FIG. 2A). As an example, the offset distance d₃ may be in a range from 2 mm to 6 mm. In general, the offset distance d₃ should be selected such that when the leaflets are attached to the frame 102 via the commissure openings 146, the free edge portions (for example, outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in other examples, the frame 102 can comprise more or less than six support posts 107 and/or more or less than three commissure support structures 144.

The inflow end portion 138 of each support post 107 can comprise an extension 154 (show as a cantilevered strut in FIGS. 2A and 2B) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures 156.

As an example, each extension 154 can have an aperture 156 (FIG. 2A) or other features to receive a suture or other attachment material for connecting an adjacent inflow edge portion 160 of a leaflet 158 (FIG. 1A), the outer skirt 103 (in FIG. 1B), the connecting skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the connecting skirt 125 and/or the inflow edge portions 160 of the leaflets 158 that have already been secured to frame 102 as well as to the extensions 154 of the frame by sutures 129. The outflow edge of the outer skirt 103 (the upper edge in FIG. 1B) can be attached to selected struts with stitches 137. In implementations where the prosthetic valve includes an inner skirt, the inflow edge of the inner skirt can be secured to the inflow edge portions 160 before securing the cusp edge portions to the frame so that the inner skirt will be between the leaflets and the inner surface of the frame. After the inner skirt and leaflets are secured in place, then the outer skirt can be mounted around the frame as described above.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In other examples, the frame 102 can be formed from a self-expandable material (for example, Nitinol). When the frame 102 is formed from a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame.

As introduced above, the threaded rods 126 can removably couple the prosthetic valve 100 to actuator assemblies of a delivery apparatus. Referring to FIG. 3, it illustrates an exemplary delivery apparatus 200 for delivering a prosthetic device or valve 202 (for example, prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (for example, actuator or threaded rod 126) on the prosthetic valve. For example, three actuator assemblies 208 can be provided for a prosthetic valve having three actuators. In other example, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery,

The actuator assemblies 208 can be releasably coupled to the prosthetic valve 202. For example, in the illustrated example, each actuator assembly 208 can be coupled to a respective actuator (for example, threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (for example, clockwise) can retract the sheath 216 proximally relative to the prosthetic valve 202 and rotation of the first knob 211 in a second direction (for example, counter-clockwise) can advance the sheath 216 distally. In other examples, the first knob 211 can be actuated by sliding or moving the first knob 211 axially, such as pulling and/or pushing the knob. In other examples, actuation of the first knob 211 (rotation or sliding movement of the first knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208. Rotation of the second knob 212 in a first direction (for example, clockwise) can radially expand the prosthetic valve 202 and rotation of the second knob 212 in a second direction (for example, counter-clockwise) can radially collapse the prosthetic valve 202. In other examples, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (for example, threaded rod). Once the actuator assemblies 208 are uncoupled from the prosthetic valve 202, the delivery apparatus 200 can be removed from the patient, leaving just the prosthetic valve 202 in the patient.

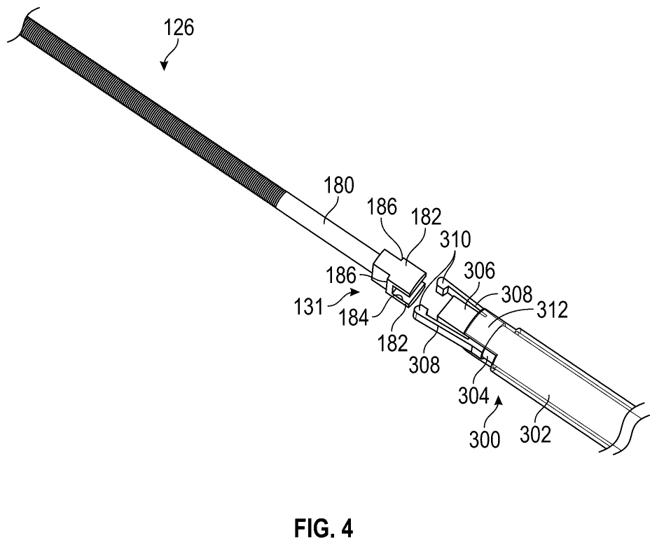

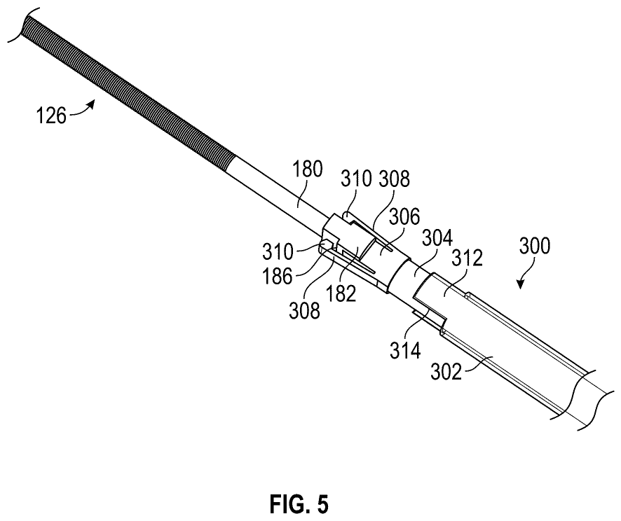

Referring to FIGS. 4-5, they illustrate how each of the threaded rods 126 of the prosthetic device 100 can be removably coupled to an exemplary actuator assembly 300 (for example, actuator assemblies 208) of a delivery apparatus (for example, delivery apparatus 200). Specifically, FIG. 5 illustrates how one of the threaded rods 126 can be coupled to an actuator assembly 300, while FIG. 4 illustrates how the threaded rod 126 can be detached from the actuator assembly 300.

As introduced above, an actuator assembly 300 can be coupled to the head portion 131 of each threaded rod 126. The head portion 131 can be included at a proximal end portion 180 of the threaded rod 126 and can extend proximally past a proximal end of the second post 124 (FIG. 2A). The head portion 131 can comprise first and second protrusions 182 defining a channel or slot 184 between them, and one or more shoulders 186. As discussed above, the head portion 131 can have a width greater than a diameter of the inner bore of the second post 124 such that the head portion 131 is prevented from moving into the inner bore of the second post 124 and such that the head portion 131 abuts the outflow end 108 of the frame 102. In particular, the head portion 131 can abut an outflow apex 119b of the frame 102. The head portion 131 can be used to apply a distally-directed force to the second post 124, for example, during radial expansion of the frame 102.

Each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 4-5 for purposes of illustration. The distal end portions of the outer sleeve 302 and driver 304 can be configured to engage or abut the proximal end of the threaded rod 126 (for example, the head portion 131) and/or the frame 102 (for example, the apex 119b). The proximal portions of the outer sleeve 302 and driver 304 can be operatively coupled to the handle of a delivery apparatus (for example, handle 204). The delivery apparatus in this example can include the same features described previously for delivery apparatus 200. In particular examples, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304. In other examples, the handle can include electric motors for actuating these components.

The distal end portion of the driver 304 can comprise a central protrusion 306 configured to extend into the slot 184 of the threaded rod 126, and one or more flexible elongated elements or arms 308 including protrusions or teeth 310 configured to be releasably coupled to the shoulders 186 of the threaded rod 126. The protrusions 310 can extend radially inwardly toward a longitudinal axis of the second actuation member 304. As shown in FIGS. 4-5, the elongated elements 308 can be configured to be biased radially outward to an expanded state, for example, by shape setting the elements 308.

As shown in FIG. 5, to couple the actuator assembly 300 to the threaded rod 126, the driver 304 can be positioned such that the central protrusion 306 is disposed within the slot 184 (FIG. 4) and such that the protrusions 310 of the elongated elements 308 are positioned distally to the shoulders 186. As the outer sleeve 302 is advanced (for example, distally) over the driver 304, the sleeve 302 compresses the elongated elements 308 they abut and/or snap over the shoulders 186, thereby coupling the actuator assembly 300 to the threaded rod 126. Thus, the outer sleeve 302 effectively squeezes and locks the elongated elements 308 and the protrusions 310 of the driver 304 into engagement with (i.e., over) the shoulders 186 of the threaded rod 126, thereby coupling the driver 304 to the threaded rod 126.

Because the central protrusion 306 of the driver 304 extends into the slot 184 of the threaded rod 126 when the driver 304 and the threaded rod 126 are coupled, the driver 304 and the threaded rod 126 can be rotational locked such that they co-rotate. So coupled, the driver 304 can be rotated (for example, using knob 212 the handle of the delivery apparatus 200) to cause corresponding rotation of the threaded rod 126 to radially expand or radially compress the prosthetic device. The central protrusion 306 can be configured (for example, sized and shaped) such that it is advantageously spaced apart from the inner walls of the outer sleeve 302, such that the central protrusion 306 does not frictionally contact the outer sleeve 302 during rotation. Though in the illustrated example the central protrusion 306 has a substantially rectangular shape in cross-section, in other examples, the protrusion 306 can have any of various shapes, for example, square, triangular, oval, etc. The slot 184 can be correspondingly shaped to receive the protrusion 306.

The outer sleeve 302 can be advanced distally relative to the driver 304 past the elongated elements 308, until the outer sleeve 302 engages the frame 102 (for example, a second post 124 of the frame 102). The distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (for example, the upper end portion) of one of the second posts 124 on opposite sides of the post 124. The engagement of the support extensions 312 with the frame 102 in this manner can counter-act rotational forces applied to the frame 102 by the rods 126 during expansion of the frame 102. In the absence of a counter-force acting against these rotational forces, the frame can tend to “jerk” or rock in the direction of rotation of the rods when they are actuated to expand the frame. The illustrated configuration is advantageous in that outer sleeves, when engaging the proximal posts 124 of the frame 102, can prevent or mitigate such jerking or rocking motion of the frame 102 when the frame 102 is radially expanded.

To decouple the actuator assembly 300 from the prosthetic device 100, the sleeve 302 can be withdrawn proximally relative to the driver 304 until the sleeve 302 no longer covers the elongated elements 308 of the driver 304. As described above, the sleeve 302 can be used to hold the elongated elements 308 against the shoulders 186 of the threaded rod 126 since the elongated elements 308 can be naturally biased to a radial outward position where the elongated elements 308 do not engage the shoulders 186 of the threaded rod 126. Thus, when the sleeve 302 is withdrawn such that it no longer covers/constrains the elongated elements 308, the elongated elements 308 can naturally and/or passively deflect away from, and thereby release from, the shoulders 186 of the threaded rod 126, thereby decoupling the driver 304 from the threaded rod 126.

The sleeve 302 can be advanced (moved distally) and/or retracted (moved proximally) relative to the driver 304 via a control mechanism (for example, knob 214) on the handle 204 of the delivery apparatus 200, by an electric motor, and/or by another suitable actuation mechanism. For example, the physician can turn the knob 214 in a first direction to apply a distally directed force to the sleeve 302 and can turn the knob 214 in an opposite second direction to apply a proximally directed force to the sleeve 302. Thus, when the sleeve 302 does not abut the prosthetic device and the physician rotates the knob 214 in the first direction, the sleeve 302 can move distally relative to the driver 304, thereby advancing the sleeve 302 over the driver 304. When the sleeve 302 does abut the prosthetic device, the physician can rotate the knob 214 in the first direction to push the entire prosthetic device distally via the sleeve 302. Further, when the physician rotates the knob 214 in the second direction the sleeve 302 can move proximally relative to the driver 304, thereby withdrawing/retracting the sleeve 302 from the driver 304.

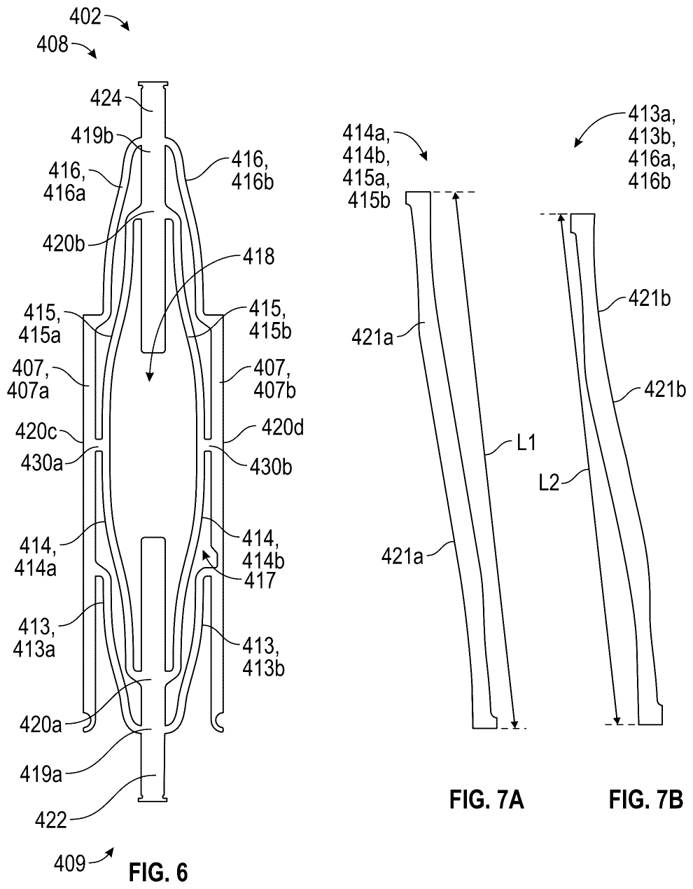

Referring now to FIG. 6, there is shown a portion of a frame 402 for a prosthetic heart valve, according to another example. The portion of the frame 402 shown in FIG. 6 includes only one outer cell 417 and one inner cell 418. It should be understood that the full frame 402 includes a plurality of pairs of outer and inner cells arranged side-by-side in the circumferential direction of the frame. A prosthetic valve can include the frame 402 and a leaflet assembly (for example, leaflet assembly 150) mounted within the frame as shown in FIGS. 1A and 1B. The prosthetic valve can include any of the components shown and described for the prosthetic valve 100, which are not repeated here for sake of brevity.

The frame 402 can comprise a plurality of support posts 407. The support posts 407 can alternatively be referred to as axial struts. Each of the plurality of support posts 407 can be oriented to extend axially, i.e., along the longitudinal axis of the frame, from an outflow end 408 of the frame 402 to an inflow end 409 of frame 402. Selected ones of the support posts 407 can comprise commissure support posts that are configured to support the commissures of the leaflet assembly. For example, the commissure support posts can include commissure openings 146 configured to receive the commissures of the leaflet assembly, as described above for the prosthetic valve 100. In certain examples, the frame 402 can include three commissure support posts for supporting three commissures of a leaflet assembly having three leaflets.

The frame 402 can further comprise a plurality of pairs of posts disposed between the plurality of support posts 407. Each pair of posts can comprise a first post 422 and a second post 424. The first post 422 may alternatively be referred to as an “inflow” or “upstream” post while the second post 424 may alternatively be referred to as an “outflow” or “downstream” post. The first post 422 and the second post 424 can be aligned with each other along the length of the frame 402. The first post 422 can extend axially from the inflow end 409 of the frame 402 toward the second post 424, and the second post 424 can extend axially from the outflow end 408 of the frame 402 toward the first post 422.

In some examples, the first post 422 and the second post 424 can be actuator posts. Each actuator post can include an inner bore configured to receive a portion of an actuator member, such as a substantially straight threaded rod or actuator (for example, threaded rod 126). In some of these examples, the first post 422 can house an internally threaded nut 127 that cooperates with the threaded rod 126. The threaded rod 126 and the nut 127 are not shown in FIG. 6 for purposes of illustration, but it should be understood that the frame 402 can include one or more pairs of threaded rods 126 and nuts 127 that are configured to radially expand and compress the frame 402, as described above in connection with the frame 102. In other examples, the first post 422 can include an internally threaded bore that engages the external threads of the threaded rod 126 that cooperate to radially expand and compress the frame 402 in lieu of a separate threaded nut 127.

In some examples, a delivery apparatus (for example, delivery apparatus 200) can configured to be releasably couple to an end portion of the second post 424 and mechanically actuate the threaded rod 126, thereby causing the frame 402 to radially expand or compress, as previously described. In examples where the delivery apparatus is releasably coupled to the end portions of the second posts 424, the second posts 424 can be referred to as “proximal posts” and the first posts 422 can be referred to as “distal posts.”

The frame 402 can further comprise multiple rows of struts that connect the first post 422 and the second post 424 to the support posts 407. In the illustrated example, the frame 402 comprises four rows of struts, including a first row of struts 413, a second row of struts 414, a third row of struts, 415, and a fourth row of struts 416. The rows of struts 413, 414, 415, and 416, the support posts 407, and the pairs of posts 422, 424 are arranged to form an outer cell 417 and an inner cell 418. For convenience, the post 407 on the left side of FIG. 6 is referred to as a left support post 407a and the post on the right side of FIG. 6 is referred to as a right support post 407b.

For each section of the frame forming a pair of outer cells 417 and inner cells 418, the first row of struts 413 can include a first or left strut 413a and a second or right strut 413b disposed at or near the inflow end 409 of the frame 402. Each of the left strut 413a and the right strut 413b can be connected at a first end to the first post 422 and can be connected at a second end to an adjacent support post 407. For example, the left strut 413a can connect the first post 422 to the left support post 407a while the right strut 413b can connect the first post 422 to the right support post 407b.

In some examples, the ends of the left strut 413a and the right strut 413b can be curved such that the end portions of these struts are perpendicular to the longitudinal axes of the first post 422, the left support post 407a, and the right support post 407b. The left strut 413a and the right strut 413b can be curved, bent, sigmoidal, or otherwise reflect a shape defined by one or more inflection points. In other examples, however, the left strut 413a and the right strut 413b can be substantially straight.

The second row of struts 414 can be disposed downstream of the first row of struts 413. For each section of the frame 402 forming a pair of inner cells 418 and outer cells 417, the second row of struts 414 can include a left strut 414a and a right strut 414b. Each of the left strut 414a and the right strut 414b can be connected at one end to the first post 422 and can be connected at another end to one of the support posts 407. For example, the left strut 414a can be connected at one end to the first post 422 and can be connected at another end to the left support post 407a, while the right strut 414b can be connected at one end to the first post 422 and can be connected at another end to the right support post 407b.

The end portions of each of the left strut 414a and the right strut 414b that are joined to the first post 422 can be curved such that the end portions are perpendicular to the longitudinal axis of the frame 402 and perpendicular to the first post 422. The end portions of each of the left strut 414a and the right strut 414b that are joined to the left support post 407a and the right support post 407b, respectively, can be curved such that the end portions are parallel to the longitudinal axis of the support posts 407.

The left strut 414a and the right strut 414b can be of the same shape, width, and/or thickness as the left strut 413a or the right strut 413b but may differ from the left strut 413a or the right strut 413b in terms of overall length. For example, in some examples of the frame 402, each of the left strut 414a and the right strut 414b can be longer than each of the left strut 413a and the right strut 413b. It has been found that the struts at the ends of the frame 402 are subject to greater forces compared to the struts closer to the middle of the frame 402 when the frame 402 is radially expanded, which can in some cases cause undesirable deformation of the struts closer to the ends of the frame 402 and/or twisting of cell columns (pairs of outer cells 417 and inner cells 418) at the ends of the frame 402. Advantageously, shortening the lengths of the left strut 413a and the right strut 413b can beneficially constrain the extent of torsion or rotation of the cell columns along the inflow end portion of the frame 402, thereby increasing the stability of the frame 402 when the prosthetic heart valve is radially expanded or compressed.

The third row of struts 415 can be disposed adjacent and downstream the second row of struts 414. The third row of struts 415 can include a left strut 415a and a right strut 415b. Each of the left strut 415a and the right strut 415b can be connected at one end to the second post 424 and connected at another end to one of the first and second support posts 407a, 407b adjacent the second post 424. For example, the left strut 415a can be connected at one end to the second post 424 and connected at another end to the left support post 407a. Likewise, the right strut 415b can be connected at one end to the second post 424 and connected at another end to the right support post 407b.

In some examples, the end portions of each of the left strut 415a and the right strut 415b joined to the second post 424 can be curved such that these end portions are perpendicular to the longitudinal axis of the second post 424. The end portions of each of the left strut 415a and the right strut 415b joined to the left support post 407a and the right support post 407b, respectively, can be curved such that these end portions are parallel to the longitudinal axes of the left support post 407a and the right support post 407b.

In some examples, the size, position, or orientation of the left strut 415a and the right strut 415b mirror the size, position, or orientation of the left strut 414a and the right strut 414b, respectively.

The left strut 415a and the right strut 415b can be of a similar length, thickness, and/or shape as the left strut 414a and the right strut 414b. Thus, in some examples, the left strut 415a and the right strut 415b can each be longer than the left strut 413a or the right strut 413b.

In some examples, the sizes, shapes, or positions of the left strut 415a and the left strut 414a are symmetrical about a lateral axis bisecting the frame 402 between the second row of struts 414 and the third row of struts 415, while the sizes, shapes, or positions of the right strut 415b and the right strut 414b are symmetrical about the lateral axis.

The fourth row of struts 416 can be disposed adjacent and downstream the third row of struts 415 such that the fourth row of struts 416 is disposed at or near the outflow end 408 of the frame 402. The fourth row of struts 416 can comprise a left strut 416a and a right strut 416b. Each of the left strut 416a and the right strut 416b can be connected at one end the second post 424 and connected at another end to one of the first or second support posts 407a, 407b adjacent the second post 424. For example, the left strut 416a can be connected at one end to the second post 424 and can be connected at another end to the left support post 407a. Likewise, the right strut 416b can be connected at one end to the second post 424 and connected at another end to the right support post 407b.

In some examples, the ends of the left strut 416a and the right strut 416b can be curved such that the end portions of these struts are perpendicular to the longitudinal axes of the second post 424, the left support post 407a, and the right support post 407b.

The left strut 416a and the right strut 416b can be curved, bent, sigmoidal, or otherwise reflect a shape defined by one or more inflection points. In other examples, however, the left strut 416a and the right strut 416b can be substantially straight.

The left strut 416a and the right strut 416b can be of a similar length, thickness, and/or shape as the left strut 413a and the right strut 413b. The left strut 416a and the right strut 416b can each be shorter than the left strut 414a, the right strut 414b, the left strut 415a, and/or the right strut 415b. It has been found that the struts at the ends of the frame 402 are subject to greater forces compared to the struts closer to the middle of the frame 402 when the frame 402 is radially expanded, which can in some cases cause undesirable deformation of the struts closer to the ends of the frame 402 and/or twisting of cell columns (pairs of outer cells 417 and inner cells 418) at the ends of the frame 402. Advantageously, shortening the lengths of the left strut 416a and the right strut 416b can beneficially constrain the extent of torsion or rotation of the cell columns along the outflow end portion of the frame 402, thereby increasing the stability of the frame 402 when the prosthetic heart valve is radially expanded or compressed.

The left strut 413a, the right strut 413b, the left strut 416a, the right strut 416b, the left support post 407a, and the right support post 407b can be arranged to form the outer cell 417. The outer cell 417 may alternatively be referred to as the “first cell” since the outer cell 417 may encompass a second inner cell (for example, the inner cell 418). Thus, the left strut 413a, the right strut 413b, the left strut 416a, the right strut 416b that form the outer cell 417 may be referred to as “outer struts.”

The outer cell 417 can form a hexagonal shape comprising an outer inflow apex 419a and an outer outflow apex 419b. The outer inflow apex 419a may alternatively be referred to as an “upstream” apex, while the outer outflow apex 419b may alternatively be referred to as a “downstream” apex. The outer inflow apex 419a can formed at a location on the first post 422 where the first post 422, the left strut 413a, and the right strut 413b converge and connect to each other. The first post 422 can extend across the outer inflow apex 419a and into the outer cell 417.

The outer outflow apex 419b can formed at a location on the second post 424 where the second post 424, the left strut 416a, and the right strut 416b converge and connect to each other. The second post 424 can extend across the outer outflow apex 419b and into the outer cell 417.

The left strut 414a, the right strut 414b, the left strut 415a, and the right strut 415b can be arranged to form an inner cell 418. The inner cell 418 may alternatively be referred to as the “second cell” since the inner cell 418 may be disposed within the outer cell 417. Thus, the left strut 414a, the right strut 414b, the left strut 415a, and the right strut 415b that form the inner cell 418 may alternatively be referred to as “inner struts.”

The inner cell 418 can form a diamond shape comprising an inner inflow apex 420a and an inner outflow apex 420b. The inner inflow apex 420a can be formed at a location on the first post 422 where the first post 422, the left strut 414a, and the right strut 414b intersect. The first post 422 can extend across the inner inflow apex 420a and into the diamond-shaped inner cell 418.

The inner outflow apex 420b can be formed at a location on the second post 424 where the second post 424, the left strut 415a, and the right strut 415b intersect. The second post 424 can extend across the inner outflow apex 420b and into the inner cell 418.

The inner cell 118 can further comprise two lateral apices: inner left apex 420c and inner right apex 420d. For convenience, the apex on the left side of FIG. 6 is referred to as the inner left apex 420c and the apex on the right side of FIG. 6 is referred to as the inner right apex 420d.

Both the inner left apex 420c and the inner right apex 420d can be disposed between the inner inflow apex 420a and the inner outflow apex 420b. The inner left apex 420c can be formed at a location where the left strut 414a and the left strut 415a intersect, while the inner right apex 420d can be at a location where the right strut 414b and the right strut 415b intersect. In the illustrated example, the adjacent ends of left strut 414a and the left strut 415a at the inner left apex 420c form a lateral connection portion 430a that intersects the left support post 407a. Similarly, the adjacent ends of right strut 414b and the right strut 415b at the inner right apex 420d form a lateral connection portion 430b that intersects the right support post 407b.

In some examples, the frame 402 can have the same construction or a substantially similar construction as the frame 102 depicted in FIGS. 1-2, except that the outer struts 413a, 413b, 416a, 416b of the frame 402 are shorter than the inner struts 414a, 414b, 415a, 415b of the frame 402.

FIG. 7A illustrates an example of one of the inner struts of the second row of struts 414 or the third row of struts 415. The term “inner strut” may refer to any of the left strut 414a, right strut 414b, left strut 415a, or right strut 415a. Each of the inner struts 414a, 414b, 415a, and 415b can comprise several curves or bends centered around one or more outer inflection points 421a. In some examples of the frame 402, the inner struts 414a, 414b, 415a, and 415b can all be the same size, length, shape, or thickness.

FIG. 7B illustrates an exemplary example of one of the outer struts of the first row of struts 413 or the fourth row of struts 416. The term “outer strut” may refer to any of the left strut 413a, the right strut 413b, the left strut 416a, and the right strut 416b. The outer struts 413a, 413b, 416a, and 416b can be straight, substantially straight, or curved. For example, the exemplary outer struts 413a, 413b, 416a, and 416b depicted in FIG. 7B can comprise several curves or bends centered around one or more inner inflection points 421b. In some examples, the inner inflection points 421b can be in the same relative locations on a strut as the outer inflection points 421a. The outer struts 413a, 413b, 416a, and 416b can be the same uniform size, shape, thickness, or form factor.

The outer struts 413a, 413b, 416a, and 416b can have a length that is less than a length of the inner struts 414a, 414b, 415a, and 415b. Moreover, the inner struts 414a, 414b, 415a, and 415b can have a straight-line length L1, defined as the length of a straight line extending from one of the strut to the other end of the strut. The outer struts 413a, 413b, 416a, and 416b can have a straight-line length L2, defined as the length of a straight line extending from one of the strut to the other end of the strut, wherein L1 is greater than L2. As noted above, the outer struts are desirably shorter than the inner struts, which advantageously better resists undesirable twisting of the cell columns during frame expansion. In some examples, the outer struts 413a, 413b, 416a, and 416b can be shorter than the inner struts 414a, 414b, 415a, and 415b but retain the same general shape as the inner struts 414a, 414b, 415a, and 415b.

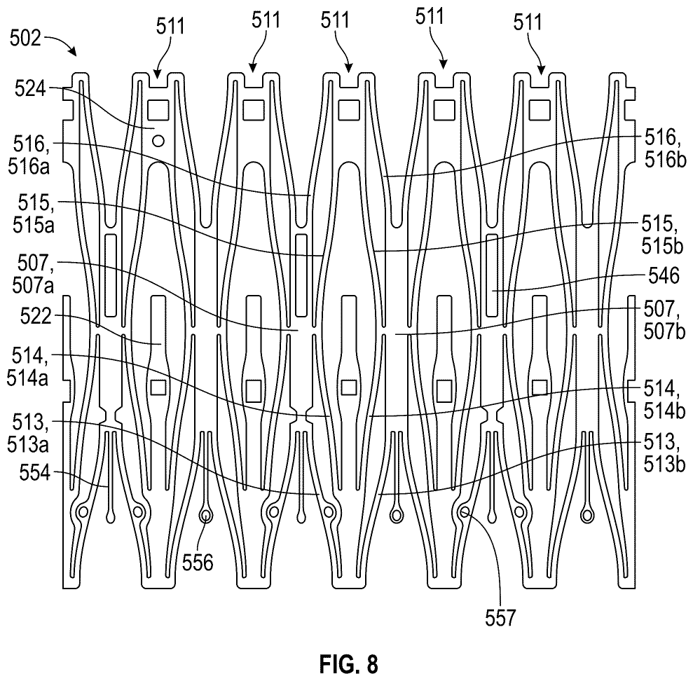

Referring now to FIG. 8, there is shown a frame 502 for a prosthetic valve, according to another example. The frame 502 can comprise a plurality of cell columns 511 arranged side-by-side around a circumference of the frame 502. Each of the cell columns 511 can comprise a first post 522, a second post 524, a plurality of support posts 507, a first row of struts 513, a second row of struts 514, a third row of struts 515, and a fourth row of struts 516. A prosthetic valve can include the frame 502 and a leaflet assembly (for example, leaflet assembly 150) mounted within the frame 502 as shown in FIGS. 1A and 1B. The prosthetic valve can include any of the components shown and described for the prosthetic valve 100, which are not repeated here for sake of brevity.

The support posts 507 can alternatively be referred to as axial struts. For convenience, the posts 507 on the left side of each of the cell columns 511 depicted in FIG. 8 are referred to as left support posts 507a and the posts on the right side of each of the cell columns 511 depicted in FIG. 8 are referred to as right support posts 507b.

The first post 522 and the second post 524 can be disposed at the inflow or upstream end of the frame 502 and the outflow or downstream end of the frame 502, respectively. The first post 522 and the second post 524 can each be oriented parallel to a longitudinal axis of the frame 502 and can be spaced apart along the length of the frame 502. In some examples, one or more pairs of the first posts 522 and the second posts 524 can include an inner bore configured to receive a portion of an actuator member (for example, threaded rod 126) configured to radially expand and compress the frame as previously described. In these examples, a delivery apparatus (for example, delivery apparatus 200) can be releasably connected to one or more of the second posts 524 to engage the actuator member. The delivery apparatus rotates the actuator member to actuate the frame 502 into an expanded or compressed configuration, as previously described.

In other examples, the first post 522 can house an internally threaded nut 127 that cooperates with the threaded rod 126. The threaded rod 126 and the nut 127 are not shown in FIG. 8 for purposes of illustration, but it should be understood that the frame 502 can include one or more pairs of threaded rods 126 and nuts 127 that are configured to radially expand and compress the frame 402, as described above in connection with the frame 102.

The left support post 507a and the right support post 507b can be adjacent the first and second posts 522, 524 to define the lateral, i.e., the left and right, sides of each of the cell columns 511. In some examples, each of the left support post 507a and the right support post 507b can further comprise an extension 554. Each extension 554 can protrude from one of the support posts 507 in an outflow or downstream direction and helps prevent portions of the prosthetic valve, such as a skirt (for example, outer skirt 103) or other components from extending into the lumen of the frame and blocking blood flow through the prosthetic valve. One or more of the extensions 554 can additionally comprise an aperture 556. The aperture 556 can be a through-hole oriented radially though the body of the extension 554. Each aperture 556 can accommodate sutures or other attachment materials for connecting leaflet 158, an outer skirt 103, a connecting skirt 125, and/or an inner skirt to the frame. The extensions 554 with the apertures 556 and the extensions 554 without the apertures 556 can be arranged on the frame 502 in an alternating pattern such that every other extension 554 includes one of the apertures 556.

Selecting ones of the support posts 507 (for example, three support posts 507) can include a commissure slot 546 or is otherwise configured to secure a respective commissure of a leaflet assembly to the frame 502, as previously described.

Each of the first row of struts 513, the second row of struts 514, a third row of struts 515, and the fourth row of struts 516 are arranged within the cell column 511 to connect the first and second posts 522, 524 to the left and right support posts 507a, 507b. Each of the rows of struts 513, 514, 515, and 516 can comprise a plurality of struts, wherein each of the struts is connected at one end to one of the first post 522 or the second post 524 and connected at another end to one of the left support post 507a or the right support post 507b. For example, the first row of struts 513 can be disposed at the inflow end of the frame 502. The first row of struts 513 can comprise a left strut 513a and a right strut 513b. The left strut 513a is connected at one end to the first post 522 and is connected at another end to the left support post 507a. The right strut 513b is connected at one end to the first post 522 and is connected at another end to the right support post 507b.

In some examples, the left strut 513a and/or the right strut 513b can include an aperture 557 for accommodating sutures or other attachment materials for securing the cusp edges of the leaflets to the frame. In the illustrated example, each of the cell columns 511 comprises an aperture 557 disposed on the left strut 513a or the right strut 513b. The cell columns 511 can be arranged such that the aperture 557 is disposed on alternating sides of adjacent cell columns 511.

The second row of struts 514 can be disposed downstream the first row of struts 513. The second row of struts 514 can comprise a left strut 514a and a right strut 514b for each cell column. The left strut 514a is connected at one end to the first post 522 and is connected at a second end to the left support post 507a. The right strut 514b is connected at one end to the first post 522 and is connected at a second end to the right support post 507b. In the illustrated example, each of the left strut 514a and the right strut 514b is longer than the left strut 513a and the right strut 513b.

The third row of struts 515 can be disposed downstream the first row of struts 513 and the second row of struts 514. The third row of struts 515 can comprise a left strut 515a and a right strut 515b for each cell column. The left strut 515a is connected at one end to the second post 524 and is connected at a second end to the left support post 507a. The right strut 515b is connected at one end to the second post 524 and is connected at another end to the right support post 507b. In the illustrated example, each of the left strut 515a and the right strut 515b is approximately the same length as left strut 514a and right strut 514b.

The fourth row of struts 516 can be disposed downstream the first row of struts 513, the second row of struts 514, and the third row of struts 515. The fourth row of struts 516 can comprise a left strut 516a and a right strut 516b for each cell column. The left strut 516a is connected at one end to the second post 524 and is connected at another end to the left support post 507a. The right strut 515b is connected at one end to the second post 524 and is connected at another end to the right support post 507b. In the illustrated example, each of the left strut 516a and the right strut 516b is shorter than the left strut 515a and the right strut 515b. In the illustrated example, each of the left strut 516a and the right strut 516b is equal in length to the left strut 513a and the right strut 513b.

In some examples, the end portions of the left fourth row strut 516a and the right fourth row strut 516b connected to the second post 524 can be curved into a “U” shape. For example, the left fourth row strut 516a and the right fourth row strut 516b can each terminate in a U-shaped bend where the left fourth row strut 516a and the right fourth row strut 516b connect to the second post 524. This bend can help minimize the crimp profile of the frame 502 when the frame 502 is in a compressed configuration.

Each cell column 511 comprises an outer cell 517 and an inner cell 518. The perimeter of the outer cell 517 is defined by the first row of struts 513, the fourth row of struts 516, and the support posts 507. In some examples, the perimeter of the outer cell 517 can form a substantially hexagonal shape. The perimeter of the inner cell 518, disposed within the perimeter of the outer cell 517, is defined by the second row of struts 514 and the third row of struts 515. In some examples, the perimeter of the inner cell 518 can form a substantially diamond shape.

Similar to the frame 402, in the illustrated frame 502, the outer struts of each cell column (struts 513a, 513b, 516a, 516b) are shorter than the inner struts of each cell column (struts 514a, 514, 515a, 515b) to improve the overall stability of the frame 502 during frame expansion. One difference between the frame 402 and the frame 502 is that in the frame 402, the ends of the outer struts (413a, 413b, 416a, 416b) that form the inflow and outflow apices 419a, 419b are offset from the ends of the first and second posts 422, 424, whereas in the frame 502, the ends of the outer struts (struts 513a, 513b, 516a, 516b) intersect the inflow and outflow ends of the first and second posts 522, 524. Also, the support posts 507 are slightly longer relative to the overall height of the frame 502 compared to the relative length of the support posts 407 to the overall height of the frame 402 to account for the relatively shorter outer struts.

In some examples, the frame 502 can have the same construction or a substantially similar construction as the frame 102 depicted in FIGS. 1-2, except that the left strut 513a, the right strut 513b, the left strut 516a, and the right strut 516b are each shorter than the left strut 514a, the right strut 514b, the left strut 515a, and the right strut 515b.

### Delivery Techniques for Disclosed Prosthetic Valves

For implanting a prosthetic valve (for example, prosthetic valve 100) within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### ADDITIONAL EXAMPLES OF THE DISCLOSED TECHNOLOGY

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. A prosthetic heart valve comprising:
a radially expandable and compressible frame comprising:
   a plurality of groups of four outer struts and two axial struts forming a plurality of hexagonal outer cells arranged side-by-side in a circumferential direction of the frame, and
   a plurality of groups of four inner struts forming a plurality of diamond-shaped inner cells, wherein each inner cell is located within a respective outer cell, wherein the outer struts are shorter than the inner struts; and
a valvular structure disposed within the frame and configured to regulate the flow of blood through the frame in one direction.
Example 2. The prosthetic heart valve of any example herein, particularly example 1, wherein the inner struts have a first uniform length.
Example 3. The prosthetic heart valve of any example herein, particularly example 2, wherein the outer struts have a second uniform length less than the first uniform length.
Example 4. The prosthetic heart valve of any example herein, particularly any one of examples 1-3, wherein the outer struts are curved.
Example 5. The prosthetic heart valve of any example herein, particularly any one of examples 1-4, wherein:
the frame comprises an inflow end and an outflow end,
each outer cell comprises an inflow apex disposed towards the inflow end and an outflow apex disposed towards the outflow end, and
for each outer cell, a first pair of two of the outer struts are connected to the inflow apex, and a second pair of two of the outer struts are connected to the outflow apex.
Example 6. The prosthetic heart valve of an example herein, particularly example 5, wherein for each outer cell, each outer strut of the first pair comprises a curved portion adjacent the inflow apex and each outer strut of the second pair comprises a curved portion adjacent the outflow apex.
Example 7. The prosthetic heart valve of any example herein, particularly example 5, wherein: each inner cell comprises an inflow apex disposed towards the inflow end and an outflow apex disposed towards the outflow end, and for each inner cell, a first pair of two of the inner struts are connected to the inflow apex of the inner cell, and a second pair of two of the inner struts are connected to the outflow apex of the inner cell.
Example 8. The prosthetic heart valve of any example herein, particularly example 7, wherein the frame comprises a plurality of pairs of first and second axially extending posts, wherein the first and second posts of each pair are axially spaced from each other, and wherein each pair of first and second posts is positioned circumferentially between two of the axial struts
Example 9. The prosthetic heart valve of any example herein, particularly example 8, wherein for each inner cell, the inner struts of the first pair of inner struts are connected to a first axially extending post of a pair of first and second posts, and the inner struts of the second pair of inner struts are connected to a second axially extending post of the pair of first and second posts.
Example 10. The prosthetic heart valve of any example herein, particularly example 9, wherein for each outer cell, the outer struts of the first pair of outer struts are connected to a first axially extending post of a pair of first and second posts, and the outer struts of the second pair of outer struts are connected to a second axially extending post of the pair of first and second posts.
Example 11. The prosthetic heart valve of any example herein, particularly any of examples 8-10, further comprising a plurality of actuator members, each extending through a pair of first and second axially extending posts.
Example 12. The prosthetic heart valve of any example herein, particularly example 11, wherein the actuator members comprise threaded rods configured to produce radially expansion of the frame from a radially compressed state to a radially expanded state upon rotation of the threaded rods.
Example 13. The prosthetic heart valve of any example herein, particularly example 12, wherein each threaded rod extends through an internally threaded nut coupled to one of the second axially extending posts.
Example 14. The prosthetic heart valve of any example herein, particularly any of examples 1-13, wherein the valvular structure comprises a plurality of leaflets forming a plurality of commissures, wherein each commissure is secured to one of the axial struts.
Example 15. A prosthetic heart valve comprising:
a radially expandable and compressible frame comprising:
   a plurality of circumferentially spaced axial struts;
   a plurality of pairs of axial posts, wherein each pair of axial posts is positioned circumferentially between two axial struts;
   a plurality of rows of angled struts, including at least a first row, a second row downstream of the first row, a third row downstream of the second row, and a fourth row downstream of the third row, wherein each angled strut is connected at one end to an axial strut and at another end to an axial post, and wherein the struts of the first and fourth rows are shorter than the struts of the second and third rows; and
      a valvular structure disposed within the frame and configured to regulate the flow of blood through the frame in one direction.
Example 16. The prosthetic heart valve of any example herein, particularly example 15, wherein the valvular structure comprises a plurality of commissures, wherein each of the commissures is connected to one of the axial struts.
Example 17. The prosthetic heart valve of any example herein, particularly any one of examples 15-16, wherein the frame further comprises a plurality of actuator members, wherein each of the actuator members extends through one of the pairs of axial posts.
Example 18. The prosthetic heart valve of any example herein, particularly example 17, wherein the actuator members comprise threaded rods configured to produce radially expansion of the frame from a radially compressed state to a radially expanded state upon rotation of the threaded rods.
Example 19. The prosthetic heart valve of any example herein, particularly example 18, wherein each threaded rod extends through an internally threaded nut coupled to an axial post.
Example 20. The prosthetic heart valve of any example herein, particularly any one of examples 15-19, wherein the second row of angled struts and the third row of angled struts form a plurality of diamond-shaped cells arranged in a circumferentially extending row of cells.
Example 21. The prosthetic heart valve of any example herein, particularly example 20, wherein the first row of angled struts, the fourth row of angled struts, and the axial struts form a plurality of hexagon-shaped cells arranged in a circumferentially extending row of cells, wherein each diamond-shaped cell is disposed within a respective hexagon-shaped cell.
Example 22. The prosthetic heart valve of any example herein, particularly any one of examples 15-21, wherein each pair of axial posts comprises an upstream axial post having an end at an inlet end of the frame and a downstream axial post having an end at an outlet end of the frame.
Example 23. A prosthetic heart valve comprising:
a radially expandable and compressible frame comprising a plurality of cell columns arranged in a circumferentially extending row of cell columns, wherein each cell column comprises an outer cell and an inner cell disposed within the outer cell, wherein each inner cell comprises four angled inner struts and each outer cell comprises four angled outer struts, wherein the outer struts are shorter than the inner struts; and
a valvular structure disposed within the frame and configured to regulate the flow of blood through the frame in one direction.
Example 24. The prosthetic heart valve of any example herein, particularly example 23, wherein:
the frame comprises a plurality of circumferentially spaced axial struts and a plurality of pairs of axial posts, wherein each pair of axial posts is positioned circumferentially between two axial struts; and
each angled outer strut is connected at one end to an axial strut and at another end to an axial post.
Example 25. The prosthetic heart valve of any example herein, particularly example 24, wherein each angled inner strut is connected at one end to an axial strut and at another end to an axial post.
Example 26. The prosthetic heart valve of any example herein, particularly any one of examples 23-25, wherein each outer cell is a hexagonal-shaped.
Example 27. The prosthetic heart valve of any example herein, particularly any one of examples 23-26, wherein each inner cell is diamond-shaped.
Example 28. The prosthetic heart valve of any example herein, particularly any one of examples 23-27, wherein each cell column extends an entire length of the frame from an inlet end of the frame to an outlet end of the frame.
Example 29. The prosthetic heart valve of any example herein, particularly any one of examples 23-28, wherein the valvular structure comprises a plurality of commissures, wherein each of the commissures is connected to one of the axial struts.
Example 30. The prosthetic heart valve of any example herein, particularly any one of examples 23-229, wherein the frame further comprises a plurality of actuator members, wherein each of the actuator members extends through one of the pairs of axial posts.
Example 31. The prosthetic heart valve of any example herein, particularly example 30, wherein the actuator members comprise threaded rods configured to produce radially expansion of the frame from a radially compressed state to a radially expanded state upon rotation of the threaded rods.
Example 32. The prosthetic heart valve of any example herein, particularly example 31, wherein each threaded rod extends through an internally threaded nut coupled to an axial post.
Example 33. The prosthetic heart valve of any example herein, particularly any one of examples 23-32, wherein the frame is cylindrical.
Example 34. The prosthetic heart valve of any example herein, particularly any one of examples 1-33, wherein the prosthetic heart valve is sterilized.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

We claim:

1. A prosthetic heart valve comprising:
a radially expandable and compressible frame comprising:
a plurality of groups of four outer struts and two axial struts forming a plurality of hexagonal outer cells arranged side-by-side in a circumferential direction of the frame, and
a plurality of groups of four inner struts forming a plurality of diamond-shaped inner cells, wherein each inner cell is located within a respective outer cell, wherein the outer struts are shorter than the inner struts; and
a valvular structure disposed within the frame and configured to regulate the flow of blood through the frame in one direction.

2. The prosthetic heart valve of claim 1, wherein the inner struts have a first uniform length.

3. The prosthetic heart valve of claim 2, wherein the outer struts have a second uniform length less than the first uniform length.

4. The prosthetic heart valve of claim 1, wherein the outer struts are curved.

5. The prosthetic heart valve of claim 1, wherein:
the frame comprises an inflow end and an outflow end,
each outer cell comprises an inflow apex disposed towards the inflow end and an outflow apex disposed towards the outflow end, and
for each outer cell, a first pair of two of the outer struts are connected to the inflow apex, and a second pair of two of the outer struts are connected to the outflow apex.

6. The prosthetic valve of claim 5, wherein for each outer cell, each outer strut of the first pair comprises a curved portion adjacent the inflow apex and each outer strut of the second pair comprises a curved portion adjacent the outflow apex.

7. The prosthetic heart valve of claim 5, wherein:
each inner cell comprises an inflow apex disposed towards the inflow end and an outflow apex disposed towards the outflow end, and
for each inner cell, a first pair of two of the inner struts are connected to the inflow apex of the inner cell, and a second pair of two of the inner struts are connected to the outflow apex of the inner cell.

8. The prosthetic heart valve of claim 7, wherein the frame comprises a plurality of pairs of first and second axially extending posts, wherein the first and second posts of each pair are axially spaced from each other, and wherein each pair of first and second posts is positioned circumferentially between two of the axial struts.

9. The prosthetic heart valve of claim 8, wherein for each inner cell, the inner struts of the first pair of inner struts are connected to a first axially extending post of a pair of first and second posts, and the inner struts of the second pair of inner struts are connected to a second axially extending post of the pair of first and second posts.

10. The prosthetic heart valve of claim 9, wherein for each outer cell, the outer struts of the first pair of outer struts are connected to a first axially extending post of a pair of first and second posts, and the outer struts of the second pair of outer struts are connected to a second axially extending post of the pair of first and second posts.

11. The prosthetic heart valve of claim 8, further comprising a plurality of actuator members, each extending through a pair of first and second axially extending posts.

12. A prosthetic heart valve comprising:
a radially expandable and compressible frame comprising:
a plurality of circumferentially spaced axial struts;
a plurality of pairs of axial posts, wherein each pair of axial posts is positioned circumferentially between two axial struts;
a plurality of rows of angled struts, including at least a first row, a second row downstream of the first row, a third row downstream of the second row, and a fourth row downstream of the third row, wherein each angled strut is connected at one end to an axial strut and at another end to an axial post, and wherein the struts of the first and fourth rows are shorter than the struts of the second and third rows; and
a valvular structure disposed within the frame and configured to regulate the flow of blood through the frame in one direction.

13. The prosthetic heart valve of claim 12, wherein the valvular structure comprises a plurality of commissures, wherein each of the commissures is connected to one of the axial struts.

14. The prosthetic heart valve of claim 12, wherein the second row of angled struts and the third row of angled struts form a plurality of diamond-shaped cells arranged in a circumferentially extending row of cells.

15. The prosthetic heart valve of claim 14, wherein the first row of angled struts, the fourth row of angled struts, and the axial struts form a plurality of hexagon-shaped cells arranged in a circumferentially extending row of cells, wherein each diamond-shaped cell is disposed within a respective hexagon-shaped cell.

16. A prosthetic heart valve comprising:
a radially expandable and compressible frame comprising a plurality of cell columns arranged in a circumferentially extending row of cell columns, wherein each cell column comprises an outer cell and an inner cell disposed within the outer cell, wherein each inner cell comprises four angled inner struts and each outer cell comprises four angled outer struts, wherein the outer struts are shorter than the inner struts; and
a valvular structure disposed within the frame and configured to regulate the flow of blood through the frame in one direction.

17. The prosthetic heart valve of claim 16, wherein:
the frame comprises a plurality of circumferentially spaced axial struts and a plurality of pairs of axial posts, wherein each pair of axial posts is positioned circumferentially between two axial struts; and
each angled outer strut is connected at one end to an axial strut and at another end to an axial post.

18. The prosthetic heart valve of claim 17, wherein each angled inner strut is connected at one end to an axial strut and at another end to an axial post.

19. The prosthetic heart valve of claim 16, wherein each cell column extends an entire length of the frame from an inlet end of the frame to an outlet end of the frame.

20. The prosthetic heart valve of claim 16, wherein the frame is cylindrical.
